(19) United States
(12) Patent Application Publication
(10) Pub. No.: US 2025/0384654 A1
(43) Pub. Date: Dec. 18, 2025

(54) LOW-FIELD MRI TEXTURE ANALYSIS

(71) Applicant: Promaxo, Inc., Oakland, CA (US)

(72) Inventors: Dang Bich THUY LE, Oakland, CA (US); Ram NARAYANAN, Oakland, CA (US); Meredith SADINSKI, Oakland, CA (US); Aleksandar NACEV, Oakland, CA (US); Srirama S. VENKATARAMAN, Oakland, CA (US)

(21) Appl. No.: 19/033,813

(22) Filed: Jan. 22, 2025

Related U.S. Application Data
(63) Continuation of application No. PCT/US2023/028471, filed on Jul. 24, 2023.
(60) Provisional application No. 63/369,301, filed on Jul. 25, 2022.

(51) Int. Cl. G06V 10/54; A61B 5/055; G01R 33/383; G01R 33/44; G01R 33/56; G06V 10/25; G06V 10/75

(52) U.S. Cl. CPC G06V 10/54; A61B 5/055; G01R 33/383; G01R 33/445; G01R 33/5602; G01R 33/5608; G06V 10/25; G06V 10/759; G06V 2201/03

(57) ABSTRACT

A system and method of identifying a region of interest using a low-field magnetic resonance imaging (MRI) system is disclosed. The method comprises obtaining a T2-weighted image from the low-field MRI system, wherein the T2-weighted image comprises a slice, annotating a first region on the slice, wherein the first region corresponds to a suspicious region, and annotating a second region on the slice, wherein the second region corresponds to a non-suspicious region. The second region comprises the same size as the first region. The method further comprises computing a first texture feature value for the first region, computing a second texture feature value for the second region, and comparing the first texture feature value to the second texture feature value.

### CROSS-REFERENCE

This application is a continuation of International Patent Application No. PCT/US2023/028471, filed Jul. 24, 2023, which claims priority to U.S. Provisional Application No. 63/369,301, filed Jul. 25, 2022, which is hereby incorporated by reference in its entirety for all purposes.

### BACKGROUND

Texture analysis of images can be used to identify different tissue types.

### SUMMARY

In one general aspect, the present disclosure provides exemplary methods of identifying a region of interest using a low-field magnetic resonance imaging (MRI) system. An exemplary method may comprise obtaining a T2-weighted image from the low-field MRI system, wherein the T2-weighted image may comprise a slice, annotating a first region on the slice, wherein the first region may correspond to a suspicious region, and annotating a second region on the slice, wherein the second region may correspond to a non-suspicious region. The second region may comprise the same size as the first region. The method may further comprise computing a first texture feature value for the first region, computing a second texture feature value for the second region, and comparing the first texture feature value to the second texture feature value.

In another aspect, the present disclosure provides low-field MRI systems. An exemplary system may comprise an array of magnets configured to generate a permanent, non-uniform BO magnetic field in a region of interest offset from the array of magnets, and a control circuit. The control circuit may be configured to generate a T2-weighted image from the single-sided, low-field MRI, identify a first region on the T2-weighted image, wherein the first region may correspond to a suspicious region, and identify a second region on the T2-weighted image. The second region may correspond to a non-suspicious region, and wherein the second region may comprise the same size as the first region. The control circuit may be configured to compute a first texture feature value for the first region, compute a second texture feature value for the second region, and compare the first texture feature value to the second texture feature value. The system may further comprise a display configured to convey the comparison of the first texture feature value to the second texture feature value.

### BRIEF DESCRIPTION OF THE DRAWINGS

The novel features of the various aspects are set forth with particularity in the appended claims. The described aspects, however, both as to organization and methods of operation, may be best understood by reference to the following description, taken in conjunction with the accompanying drawings.

FIG. 1 is a fused low-field and high-field MR image including co-registered T2-weighted images from a low-field magnetic resonance (MR) image and a high-field MR image and depicting regions annotated on the high-field MR image, according to various aspects of the present disclosure.

FIG. 2 is an array of texture maps, according to various aspects of the present disclosure.

FIGS. 3A and 3B are graphical representations of texture feature values on high-field and low-field MR images, according to various aspects of the present disclosure.

FIG. 4 is a flowchart depicting a validation study to compare Haralick texture feature values from high-field and low-field MR images, according to various aspects of the present disclosure.

FIG. 5 is a perspective view of an MRI scanner, according to various aspects of the present disclosure.

FIG. 6 is an exploded, perspective view of the MRI scanner of FIG. 5, in which the permanent magnet assembly and the gradient coil sets within the housing are exposed, according to various aspects of the present disclosure.

FIG. 7 is an elevation view of the MRI scanner of FIG. 5, according to various aspects of the present disclosure.

FIG. 8 is an elevation view of the MRI scanner of FIG. 5, according to various aspects of the present disclosure.

FIG. 9 is a perspective view of the permanent magnet assembly of the MRI scanner of FIG. 5, according to various aspects of the present disclosure.

FIG. 10 is an elevation view of the gradient coil set and the permanent magnet assembly of the MRI system shown in FIG. 5, according to various aspects of the present disclosure.

FIG. 11 is a control schematic for a single-sided MRI system, according to various aspects of the present disclosure.

FIG. 12 is a schematic of the magnetic gradient along the Z axis, according to various aspects of the present disclosure.

The accompanying drawings are not intended to be drawn to scale. Corresponding reference characters indicate corresponding parts throughout the several views. For purposes of clarity, not every component may be labeled in every drawing. The exemplifications set out herein illustrate certain embodiments of the invention, in one form, and such exemplifications are not to be construed as limiting the scope of the invention in any manner.

### DETAILED DESCRIPTION

The following international patent applications are incorporated by reference herein in their respective entireties:
International Application No. PCT/US2020/018352, titled SYSTEMS AND METHODS FOR ULTRALOW FIELD RELAXATION DISPERSION, filed Feb. 14, 2020, now International Publication No. WO2020/168233;
International Application No. PCT/US2020/019530, titled SYSTEMS AND METHODS FOR PERFORMING MAGNETIC RESONANCE IMAGING, filed Feb. 24, 2020, now International Publication No. WO2020/172673;
International Application No. PCT/US2020/019524, titled PSEUDO-BIRDCAGE COIL WITH VARIABLE TUNING AND APPLICATIONS THEREOF, filed Feb. 24, 2020, now International Publication No. WO2020/172672;
International Application No. PCT/US2020/024776, titled SINGLE-SIDED FAST MRI GRADIENT FIELD COILS AND APPLICATIONS THEREOF, filed Mar. 25, 2020, now International Publication No. WO2020/198395;
International Application No. PCT/US2020/024778, titled SYSTEMS AND METHODS FOR VOLUMETRIC ACQUISITION IN A SINGLE-SIDED MRI SYSTEM, filed Mar. 25, 2020, now International Publication No. WO2020/198396;
International Application No. PCT/US2020/039667, title SYSTEMS AND METHODS FOR IMAGE RECONSTRUCTIONS IN MAGNETIC RESONANCE IMAGING, filed Jun. 25, 2020, now International Publication No. WO2020/264194
International Application No. PCT/US2021/014628, titled MRI-GUIDED ROBOTIC SYSTEMS AND METHODS FOR BIOPSY, filed Jan. 22, 2021, now International Publication No. WO2021/150902;
International Application No. PCT/US2021/018834, titled RADIO FREQUENCY RECEPTION COIL NETWORKS FOR SINGLE-SIDED MAGNETIC RESONANCE IMAGING, filed Feb. 19, 2021, now International Publication No. WO2021/168291;
International Patent Application No. PCT/US2021/021464, titled PHASE ENCODING WITH FREQUENCY SWEEP PULSES FOR MAGNETIC RESONANCE IMAGING IN INHOMOGENEOUS MAGNETIC FIELDS, filed Mar. 9, 2021, now International Publication No. WO2021/183484;
International Patent Application No. PCT/US2021/021461, titled PULSE SEQUENCES AND FREQUENCY SWEEP PULSES FOR SINGLE-SIDED MAGNETIC RESONANCE IMAGING, filed Mar. 9, 2021, now International Publication No. WO/2021183482;
International Patent Application No. PCT/US2021/021464, titled PHASE ENCODING WITH FREQUENCY SWEEP PULSES FOR MAGNETIC RESONANCE IMAGING IN INHOMOGENEOUS MAGNETIC FIELDS, filed Mar. 9, 2021, now International Publication No. WO2021/183484; and
International Patent Application No. PCT/US2022/071924, titled LOCALIZATION GUIDE AND METHOD FOR MRI GUIDED PELVIC INTERVENTIONS, filed Apr. 26, 2022, is also incorporated by reference herein in its entirety.

The following United States provisional patent applications are incorporated by reference herein in their respective entireties:
U.S. Provisional Patent Application No. 62/806,664, titled SYSTEMS AND METHODS FOR ULTRALOW FIELD RELAXATION DISPERSION, filed Feb. 15, 2019;
U.S. Provisional Patent Application No. 62/809,503, titled PSEUDO-BIRDCAGE COIL WITH VARIABLE TUNING AND APPLICATIONS THEREOF, filed Feb. 22, 2019;
U.S. Provisional Patent Application No. 62/823,521, titled SINGLE-SIDED FAST MRI GRADIENT FIELD COILS AND APPLICATIONS THEREOF, filed Mar. 25, 2019;
U.S. Provisional Patent Application No. 62/866,533, titled SYSTEMS AND METHODS FOR IMAGE RECONSTRUCTION IN MAGNETIC RESONANCE IMAGING, filed Jun. 15, 2019;
U.S. Provisional Patent Application No. 62/965,070, titled GUIDED ROBOTIC SYSTEM, METHODS AND APPARATUS FOR BIOPSY, filed Jan. 23, 2020;
U.S. Provisional Patent Application No. 62/979,332, titled SYSTEM AND METHODS FOR UTILIZING A RADIO FREQUENCY RECEIVE NETWORK FOR SINGLE-SIDED MAGNETIC RESONANCE IMAGING, filed Feb. 20, 2020;
U.S. Provisional Patent Application No. 62/987,286, titled SYSTEMS AND METHODS FOR ADAPTING DRIVEN EQUILIBRIUM FOURIER TRANSFORM FOR SINGLE-SIDED MRI, filed Mar. 9, 2020;
U.S. Provisional Patent Application No. 62/987,292, titled SYSTEMS AND METHODS FOR LIMITING K-SPACE TRUNCATION IN A SINGLE-SIDED MRI SCANNER, filed Mar. 9, 2020;
U.S. Provisional Patent Application No. 62/823,511, titled SYSTEMS AND METHODS FOR VOLUMETRIC ACQUISITION IN A SINGLE-SIDED MRI SCANNER, filed Mar. 25, 2019;
U.S. Provisional Patent Application No. 63/180,013, titled INTERVENTIONAL LOCALIZATION GUIDE AND METHOD FOR MRI GUIDED PELVIC INTERVENTIONS, filed Apr. 26, 2021;
U.S. Provisional Patent Application No. 63/266,383, titled RELAXATION-BASED MAGNETIC RESONANCE THERMOMETRY WITH A LOW-FIELD SINGLE-SIDED MRI SCANNER, filed Jan. 4, 2022; and
U.S. Provisional Patent Application No. 63/367,787, titled BIOPSY DEVICES AND METHODS, filed Jul. 6, 2022;

The following United States patent applications are incorporated by reference herein in their respective entireties:
U.S. Patent Application Publication No. 2018/0356480, titled UNILATERAL MAGNETIC RESONANCE IMAGING SYSTEM WITH APERTURE FOR INTERVENTIONS AND METHODOLOGIES FOR OPERATING SAME, published Dec. 13, 2018;
U.S. Patent Application Publication No. 2022/0146613, titled SYSTEMS AND METHODS FOR ULTRALOW FIELD RELAXATION DISPERSION, published May 12, 2022;
U.S. Patent Application Publication No. 2022/0043084, titled PSEUDO-BIRDCAGE COIL WITH VARIABLE TUNING AND APPLICATIONS THEREOF, published Feb. 10, 2022;
U.S. Patent Application Publication No. 2022/0113361, titled SYSTEMS AND METHODS FOR PERFORMING MAGNETIC RESONANCE IMAGING, published Apr. 14, 2022;
U.S. Patent Application Publication No. 2022/0091207, titled SINGLE-SIDED FAST MRI GRADIENT FIELD COILS AND APPLICATIONS THEREOF, published Mar. 24, 2022;
U.S. patent application Ser. No. 17/596,610, titled SYSTEMS AND METHODS FOR IMAGE RECONSTRUCTION IN MAGNETIC RESONANCE IMAGING, filed Dec. 14, 2021;
U.S. patent application Ser. No. 17/596,610, titled SYSTEMS AND METHODS FOR IMAGE RECONSTRUCTION IN MAGNETIC RESONANCE IMAGING, filed Dec. 14, 2021; and
U.S. patent application Ser. No. 17/660,709, titled INTERVENTIONAL LOCALIZATION GUIDE AND METHOD FOR MRI GUIDED PELVIC INTERVENTIONS, filed Apr. 26, 2022.

The following United States design applications are incorporated by reference herein in their respective entireties:
U.S. Design application Ser. No. 29/681,014, titled ANALYTICAL DEVICE, filed Feb. 21, 2019, now U.S. Design Patent D895,803, issued Sep. 8, 2020;
U.S. Design application Ser. No. 29/744,371, titled ANALYTICAL DEVICE, filed Jul. 28, 2020, now U.S. Design Patent D942,012, issued Jan. 25, 2022; and
U.S. Design application Ser. No. 29/790,895, titled ANALYTICAL DEVICE, filed Dec. 20, 2021.

Before explaining various aspects of an MRI system and method in detail, it should be noted that the illustrative examples are not limited in application or use to the details of construction and arrangement of parts illustrated in the accompanying drawings and description. The illustrative examples may be implemented or incorporated in other aspects, variations, and modifications, and may be practiced or carried out in various ways. Further, unless otherwise indicated, the terms and expressions employed herein have been chosen for the purpose of describing the illustrative examples for the convenience of the reader and are not for the purpose of limitation thereof. Also, it will be appreciated that one or more of the following-described aspects, expressions of aspects, and/or examples, can be combined with any one or more of the other following-described aspects, expressions of aspects, and/or examples.

Radiomics is the quantitative extraction and analysis of minable data from medical images. It may be used to identify different types of tissue. For example, it may be used to detect and categorize prostate lesions. Radiomics involves extraction of quantitative features, i.e. radiomic features, from radiological images that typically cannot be seen by a radiologist's naked eye. For example, radiomic features can include texture features like Energy, Entropy, Correlation, Homogeneity, and Inertia. Haralick texture features are calculated from a gray level co-occurrence matrix (GLCM), which is a matrix that is defined over an image having a distribution of co-occurring pixel values (grayscale values, or colors) at a given offset. GLCM is used as an approach to texture analysis with various applications especially in medical image analysis; features generated using this technique are usually called Haralick features, after Robert Haralick. Haralick features extract frequencies of local spatial variations in signal intensity in an image and quantify the pixel relationships within regions of interest in the image. For example, Haralick features can be determined by determining/counting the co-occurrence of neighboring gray levels in an image.

Haralick texture analysis of prostate MRIs have only been studied for cancer detection in connection with high-field MRIs. High-field MRIs have an electromagnetic field that is greater than 1.5 T. Typically, high-field MRIs have an electromagnetic field between 1.5 T and 3 T. The following articles are incorporated by reference herein in their respective entireties:
Cutaia, Giuseppe et al. “Radiomics and Prostate MRI: Current Role and Future Applications.” Journal of imaging vol. 7,2 34. 11 Feb. 2021;
Nketiah, Gabriel A et al. “Utility of T2-weighted MRI texture analysis in assessment of peripheral zone prostate cancer aggressiveness: a single-arm, multicenter study.” Scientific reports vol. 11,1 2085. 22 Jan. 2021;
Baek, Tae Wook et al. “Texture analysis on bi-parametric MRI for evaluation of aggressiveness in patients with prostate cancer.” Abdominal radiology (New York) vol. 45,12 (2020): 4214-4222;
Niu, Xiang-Ke et al. “Clinical Application of Biparametric MRI Texture Analysis for Detection and Evaluation of High-Grade Prostate Cancer in Zone-Specific Regions.” AJR. American journal of roentgenology vol. 210,3 (2018): 549-556; and
Wibmer, Andreas et al. “Haralick texture analysis of prostate MRI: utility for differentiating non-cancerous prostate from prostate cancer and differentiating prostate cancers with different Gleason scores.” European radiology vol. 25,10 (2015): 2840-50.

In various instances, low-field MRIs are preferable to high-field MRIs, as further described herein. For example, low-field MRI systems can have a smaller footprint than high-field MRI systems and/or can require reduced shielding requirements, which can be preferable in certain instances. Moreover, low-field MRIs can be more open-concept that high-field MRIs. For example, single-sided, low-field MRIs can provide an improved patient experience and allow improved accessibility by a clinician and/or surgical robot. International Application No. PCT/US2021/014628, titled MRI-GUIDED ROBOTIC SYSTEMS AND METHODS FOR BIOPSY, filed Jan. 22, 2021, which is incorporated by reference herein its entirety, describes MRI-guided biopsy procedures, for example.

However, low-field MR images have distinct differences from high-field MR images. For example, T2 contrast is impacted by field strength. Noise patterns are also different between low-field and high-field MR images. More specifically, noise in high-field MRI scanners is typically dominated by the object being imaged, with additional noise from hardware. At low-fields, object noise may be negligible and the overall noise may be dominated by hardware components, such as the RF coils and spectrometers.

In various aspects of the present disclosure, Haralick texture analysis can be utilized to differentiate cancerous and non-cancerous regions in images from a low-field, single-sided MRI system. For example, the image processing for Haralick texture analysis can be applied to low-field images from a low-field, single-sided MRI as follows. Regions of interest (ROI) suspicious for cancer (such as suspicious/cancerous regions in the prostate) can be annotated on T2-weighted images from the low-field, single-sided MRI. For each cancerous ROI, a secondary ROI of identical size can be drawn on the same slice in a clinically non-suspicious region (e.g. such as non-cancerous tissue also in the prostate), which can be presumed to be normal, non-cancerous tissue. The images can be normalized and rescaled into n gray level bins, where n is between 4 and 256. For each ROI, the GLCM can be computed in four or eight directions on transverse, 2D slices. Four Haralick texture maps (Contrast, Energy, Correlation, and Homogeneity) can be created to assess the pixel-to-pixel relationship in suspicious and non-suspicious regions by calculating texture measures within a local neighborhood using a sliding window technique over the entire prostate region of the image, and then averaging the values of the resulting texture maps in suspicious and non-suspicious ROIs.

In other instances, Haralick texture measures can be extracted within respective ROIs (cancerous and non-suspicious regions).

Application of Haralick texture analysis to low-field MRI images can differentiate between suspicious (e.g. cancerous) and non-suspicious (e.g. presumed to be non-cancerous) ROIs. More specifically, the values of texture measures within a suspicious ROI compared to those of a non-suspicious ROI demonstrate a consistent relationship. For example, Energy and Homogeneity texture features can be elevated within suspicious regions compared to non-suspicious regions, while Contrast and Correlation texture features can be reduced within suspicious regions compared to non-suspicious regions, as further described herein.

The foregoing texture analysis can allow differentiation and characterization of tissue using low-field MRIs.

In one aspect of the present disclosure, T2-weighted images from low-field MRIs can be analyzed for texture features indicative of cancerous tissue.

In one aspect of the present disclosure, cancerous region(s) in the prostate can be distinguished from normal tissue by applying Haralick texture analysis to low-field, T2-weighted images.

In various aspects of the present disclosure, low-field MRI images can be analyzed for texture features using a GLCM where the gray level includes between four and 256 bins, the window size is (5, 5)-(49, 49) pixel, and the sliding window stride is one to ten pixels.

In one aspect, for both 3T and low-field datasets, images can be normalized and rescaled into L gray level bins, using following formula:

$$I = \frac{I - min ( l )}{max ( l ) - min ( l )} * L$$

Where I is the image and L is the gray level bins. In various instances, L can be 64, i.e. the image is normalized and rescaled into 64 gray level bins.

In one exemplary application of the present disclosure, Haralick texture analysis can be applied to differentiate suspicious prostrate lesions from normative tissue on low-field MRIs. Prostate cancer is the second most commonly diagnosed cancer and the fourth leading cause of cancer mortality in men. For accurate diagnosis and timely and effective treatment, it may be essential to identify suspicious regions accurately for acquiring a biopsy. MR images have been used in targeted prostate biopsy to pre-assess whether patients should have a prostate biopsy, as well as where to take the biopsy. Pre-procedure MR images with annotations, assigned a degree of suspicion (e.g. with a Prostate Imaging Reporting and Data System (PI-RADS), version 2 scoring system) can be cognitively or electronically co-registered to real-time ultrasound images to provide guidance during a biopsy, for example. However, in some instances, fusion biopsy with ultrasound images has demonstrated significant disadvantages such as gland deformation, steep learning curve, and registration inaccuracies, which limit its adoption.

Alternatively, a low-field MRI system, such as the low-field MRI system provided by Promaxo Inc. (Oakland, CA), can provide an office-based, open single-sided scanner that operates at a low, non-uniform BO field (58-74 mT) with non-linear x-and y-axis gradients and a permanent, built-in z-gradient. The system can be used for guiding transperineal prostate biopsy interventions. In various instances, the low-field MRI scanner can acquire images along the transverse direction without transrectal probes. Moreover, the patient can be positioned similar to high-field (1.5T-3T) MRIs. Exemplary single-sided, low-field MRI systems are further described herein. During the biopsy with guidance from the low-field MRI system, the high-field, T2-weighted MR images with annotations by a radiologist can be overlaid on the low-field, T2-weighted images. The low-field and high-field images can be fused together to directly target abnormal regions seen and/or annotated on the high-field MR images.

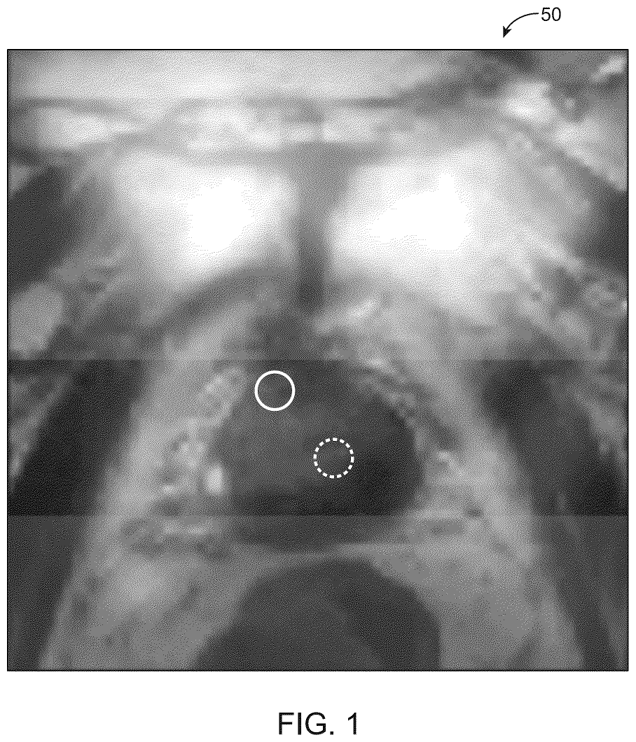

As an example, a suspicious ROI in the prostate can be annotated on a 3T, T2-weighted image by a radiologist and assigned a Prostate Imaging Reporting and Data System (PI-RADS) score. The 3T MR image volumes and low-field MR image volumes can be rigidly co-registered and the radiologist-performed annotations propagated from the 3T image to the co-registered, low-field image. Then, for each cancerous ROI, a secondary ROI of identical size can be drawn on the same slice in a clinically non-suspicious region of the prostate presumed to be normal tissue. An exemplary co-registered T2-weighted image 50 from 3T and low-field MRs are shown in FIG. 1. In FIG. 1, the patient has Gleason score 4+5 prostate cancer. A cancerous lesion is indicated with the circle on the right and a non-suspicious region with the same radius is marked with the circle on the left.

Regardless of what imaging modality is used for guidance in a targeted biopsy, it can be challenging to localize where to take a biopsy because clinical analysis of MR images have been largely qualitative, e.g. with cancerous regions being identified and annotated by radiologists. However, in various instances, texture analysis can be applied to medical images, as further described herein. Image texture analysis is a technique to extract frequencies of local spatial variations in signal intensity to quantifying pixel relationships within regions of interest and capturing image patterns that may be (and usually are) indistinguishable to the human eye. A common image texture analysis technique is Haralick texture analysis, as further described herein, which can be applied to quantitatively characterize breast cancer, colon cancer, and rectal cancer, for example. Moreover, as further described herein, Haralick texture analysis has been studied for prostate cancer detection on T2-weighted, 3T MR images.

Haralick features of Energy, Correlation, Contrast, and Homogeneity can be extracted from MR images of the prostate, using one or more methods. A first method involves extracting Haralick texture measures within respective ROIs (cancerous and non-suspicious regions). A second method involves creating four texture maps (Contrast, Energy, Correlation, and Homogeneity) by calculating texture measures within a local neighborhood using a sliding window technique over the entire prostate region of the image then averaging the values of the resulting texture maps in cancerous and non-suspicious ROIs.

The evaluated texture features can demonstrate consistency in texture measures for cancerous regions compared to non-suspicious within ROIs from the same patient, where Energy and Homogeneity were elevated while Contrast and Correlation are reduced within cancerous regions compared to non-suspicious regions. Consequently, several Haralick texture features show promise for cancer detection in low-field T2-weighted MR images.

Haralick texture analysis utilizes GLCM, a two-dimensional histogram that captures the frequency of co-occurrence of two pixel intensities at a certain offset. The GLCM considers the relationship between groups of two pixels in the original image, called the reference pixels and the neighbor pixels. The values in GLCM are the counts of frequencies of the neighboring pairs of image pixel values. GLCM can be symmetrical for the best performance of texture calculations, and for overcoming problem of the window edge pixels. In this context, symmetry means that the matrix counts each reference pixel with the neighbor to both its right and its left so each pixel pair is counted twice, once forward and once backward, interchanging reference and neighbor pixels for the second count. The GLCM may then be normalized by dividing by the total number of accumulated co-occurrences. In normalized symmetrical GLCM, the diagonal elements all represent pixel pairs with no grey level difference and the farther away from the diagonal, the greater the difference between pixel grey levels.

Texture measures are the various single values used to summarize the normalized symmetrical GLCM in different way. Robert Haralick proposed fourteen different measures and these texture features are correlated with each other. They can be divided into three groups—Contrast group, Orderliness group, and Description Statistics group—that are independent of each other. The Contrast group includes Contrast, Dissimilarity, and Homogeneity, using weights related to the distance from the GLCM diagonal. The Orderliness group measures how often a given pair of two grey levels occur within a window. Orderliness features include Angular Second Moment (ASM), Energy, Maximum Probability, and Entropy. The Descriptive Statistics group includes GLCM Mean, Variance, and Correlation. Contrast, Homogeneity, Energy, and Correlation are useful for distinguishing cancer by outcomes in certain instances.

In various instances, the following equations can be utilized to calculate these measures:

Normalization equation:

$${P}_{i , j} = \frac{{V}_{i , j}}{{\sum }_{i , j = 0}^{N - 1} {V}_{i , j}}$$

Where i, j are the row and column number. V is the value of cell i, j of the image window. And Pij is the value recorded for the cell i,j of normalized GLCM.

$$\sqrt{{\sum }_{i , j = 0}^{N - 1} {P}_{i , {j}^{2}}} \qquad Energy$$

$${\sum }_{i , j = 0}^{N - 1} {{P}_{i , j} ( i - j )}^{2} \qquad Contrast$$

$${\sum }_{i , j = 0}^{N - 1} \frac{{P}_{i , j}}{1 + {( i - j )}^{2}} \qquad Homogeneity$$

$${\sum }_{i , j = 0}^{N - 1} {P}_{i , j} [ \frac{( i - {μ}_{i} ) ( j - {μ}_{j} )}{\sqrt{( {σ}_{i}^{2} ) ( {σ}_{j}^{2} )}} ] . \qquad Correlation$$

Where μ is mean:

$${μ}_{i} = {\sum }_{i , j = 0}^{N - 1} {iP}_{i , j}$$

And σ is variance:

$${σ}_{i} \sqrt{{\sum }_{i , j = 0}^{N - 1} {{P}_{i , j} ( i - {μ}_{i} )}^{2}}$$

For a symmetrical GLCM, the mean and variance calculated using i or j gives the same results.

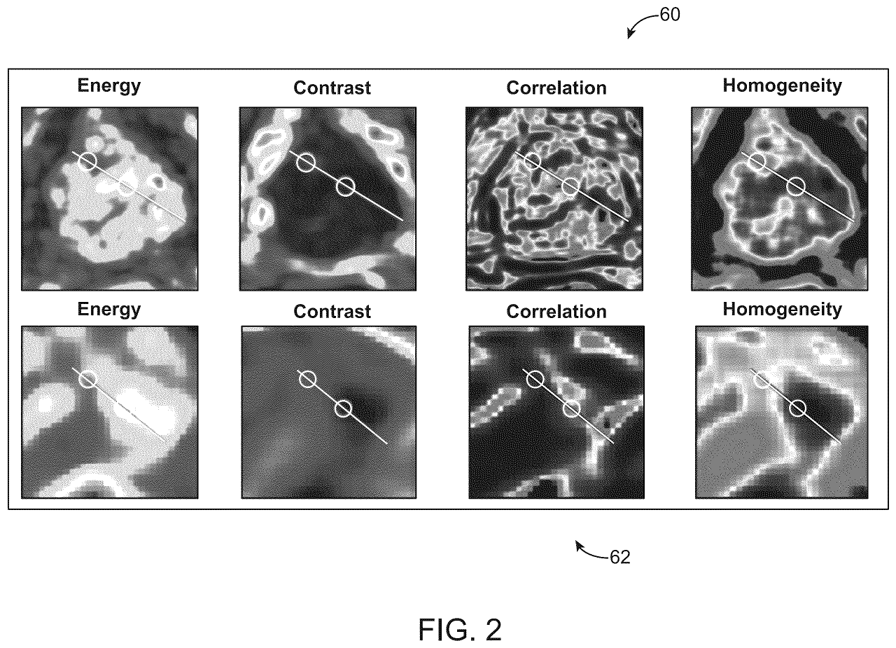

A texture image or texture map can then be created. Exemplary texture maps are shown in FIG. 2. In FIG. 2, the upper row 60 depicts texture maps from a high-field MR image and the lower row 62 depicts texture maps from a low-field MR image. The cancerous region is indicated with the circle on the right and a non-suspicious region with the same radius is marked with the circle on the left.

To see the variant pixel-to-pixel relationships in various parts of the image, the texture measure can be calculated using the GLCM derived from a small area on the image at a time. The texture measure in another small area can then be calculated until the entire image has been covered. Creating texture image this way can help to quantitatively assess how the pixel relationships vary in different regions.

In various instances, the following steps can be followed to create the texture map: Step One, decide on the window size, which is the small area for filling in the GLCM and doing the texture measure calculation. The window size is a square and has an odd number of pixels on a side. Step Two, place the window in the first position over top left of the image. Step Three, create the GLCM for this window and normalize. Step Four, calculate the texture measure of choice, which is the single number representing the entire window. This number is put in the place of the center pixel of the window. Step Five, move the window over the predefined distance (usually one pixel) and repeat Steps Three and Four. Step Six, continue with all possible window positions until the texture map is done.

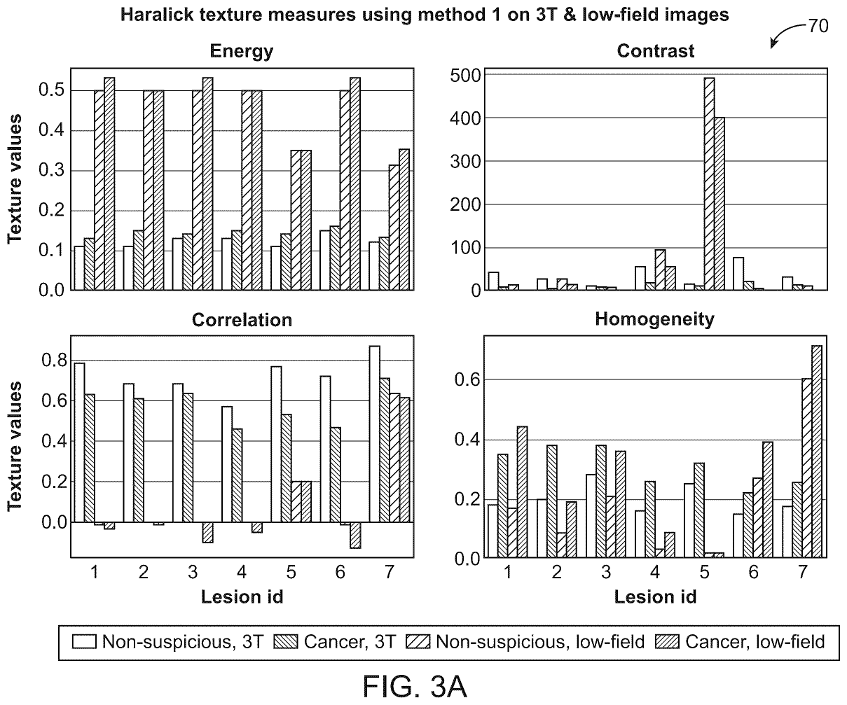

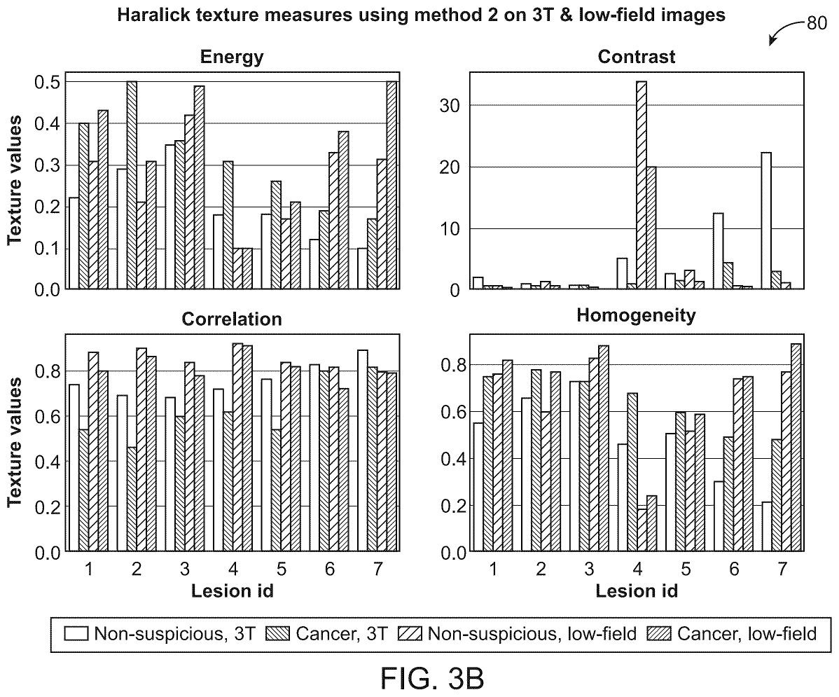

In various instances, Haralick texture measures on high-field and low-field MR images can be graphed and, in certain instances, can be compared. For example, referring to FIG. 3A and 3B, Energy, Contrast, Correlation, and Homogeneity texture values are depicted in graphical representations 70, 80, respectively, for comparing high-field and low-field MR images. The texture values were calculated according to different methods in FIG. 3A and 3B. Nonetheless, using both methods, for cancerous and non-suspicious regions from the same patient, Contrast and Correlation texture values were lower, while Energy and Homogeneity texture values were higher in cancerous regions than in non-suspicious regions in both high-field and low-field MR images.

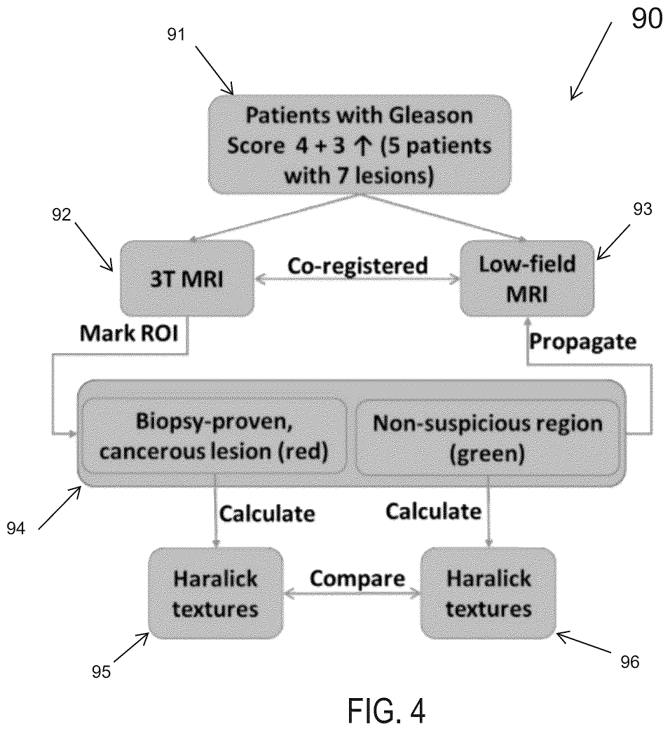

Referring now to FIG. 4, a flowchart 90 depicting a validation study technique for comparing Haralick features calculated from 3T MR images and low-field MR images is shown. In this example, the dataset included patients with Gleason Score 4+3 prostate cancer (91). The example study included five patients with seven total lesions. The patients underwent a 3T MRI scan (92) and a low-field (58mT-74mT) MRI scan of their prostate (93). The suspicious ROI in the prostate was annotated on a 3T, T2-weighted image by a radiologist and assigned the Prostate Imaging Reporting and Data System (PI-RADS) score. The 3T MR image volumes and low-field MR image volumes were rigidly co-registered and the radiologist-performed annotations from the 3T MR image were propagated to the co-registered, low-field MR image (94). Then, for each suspicious ROI, a secondary ROI of identical size was drawn on the same slice in a clinically non-suspicious region of the prostate presumed to be normal tissue. Haralick texture features were calculated from both the 3T MR image (95) and the low-field MR image (96) in the suspicious ROI and non-suspicious ROI. Though the numerical values of the Haralick texture features were different from the different images, the relative texture values demonstrated patterns. More specifically, referring again to FIGS. 3A and 3B, Contrast and Correlation texture values were lower, while Energy and Homogeneity texture values were higher in cancerous regions than in non-suspicious regions in both high-field and low-field MR images.

An exemplary low-field, single-sided MRI system is further described herein. In accordance with various aspects, an MRI system is provided that can include a unique imaging region that can be offset from the face of a magnet. Such offset and single-sided MRI systems are less restrictive as compared to traditional MRI scanners. In addition, this form factor can have a built-in or inherent magnetic field gradient that creates a range of magnetic field values over the region of interest. In other words, the inherent magnetic field can be inhomogeneous. The inhomogeneity of the magnetic field strength in the region of interest for the single-sided MRI system can be more than 200 parts per million (ppm). For example, the inhomogeneity of the magnetic field strength in the region of interest for the single-sided MRI system can between 200 ppm and 200,000 ppm. In various aspects of the present disclosure, the inhomogeneity in the region of interest can be greater than 1,000 ppm and can be greater than 10,000 ppm. In one instance, the inhomogeneity in the region of interest can be 81,000 ppm.

The inherent magnetic field gradient can be generated by a permanent magnet within the MRI scanner. The magnetic field strength in the region of interest for the single-sided MRI system can be less than 1 Tesla (T), for example. For example, the magnetic field strength in the region of interest for the single-sided MRI system can be less than 0.5 T. In other instances, the magnetic field strength can be greater than 1 T and may be 1.5 T, for example. This system can operate at a lower magnetic field strength as compared to typical MRI systems allowing for a relaxation on the RX coil design constraints and/or allowing for additional mechanisms, like robotics, for example, to be used with the MRI scanner. Exemplary MRI-guided robotic systems are further described in International Application No. PCT/US2021/014628, titled MRI-GUIDED ROBOTIC SYSTEMS AND METHODS FOR BIOPSY, filed Jan. 22, 2021, for example.

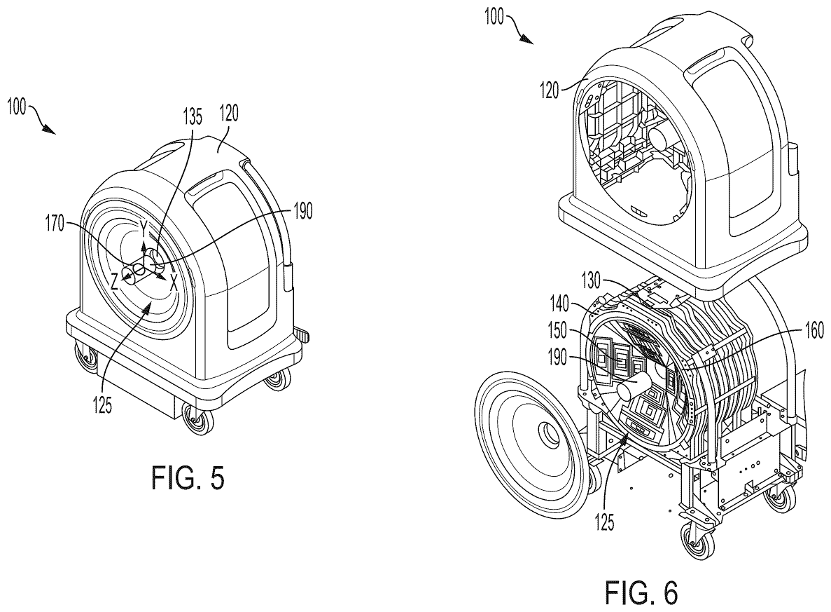

FIGS. 5-11 depict an MRI scanner 100 and components thereof. As shown in FIGS. 5 and 6, the MRI scanner 100 includes a housing 120 having a face or front surface 125, which is concave and recessed. In other aspects, the face of the housing 120 can be flat and planar. The front surface 125 can face the object being imaged by the MRI scanner. As shown in FIGS. 5 and 6, the housing 120 includes a permanent magnet assembly 130, an RF transmission coil (TX) 140, a gradient coil set 150, an electromagnet 160, and a RF reception coil (RX) 170. In other instances, the housing 120 may not include the electromagnet 160. Moreover, in certain instances, the RF reception coil 170 and the RF transmission coil 140 can be incorporated into a combined Tx/Rx coil array. In various instances, the MRI scanner 100 is a single-sided scanner and the various components, e.g. the permanent magnet assembly 130, the RF transmission coil (TX) 140, the gradient coil set 150, the electromagnet 160, and the RF reception coil (RX) 170, are positioned on the same side of the field of view.

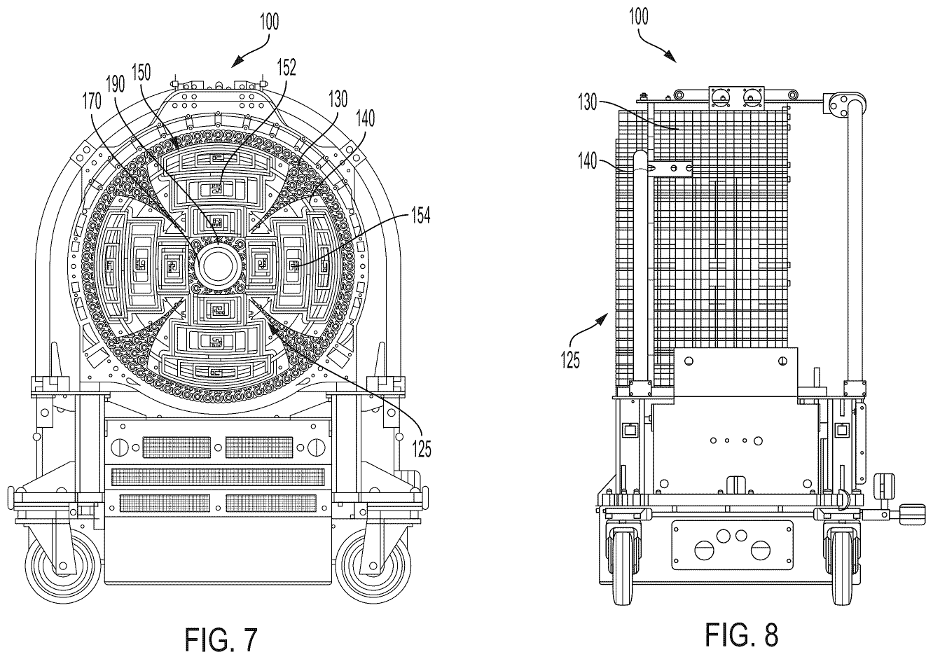

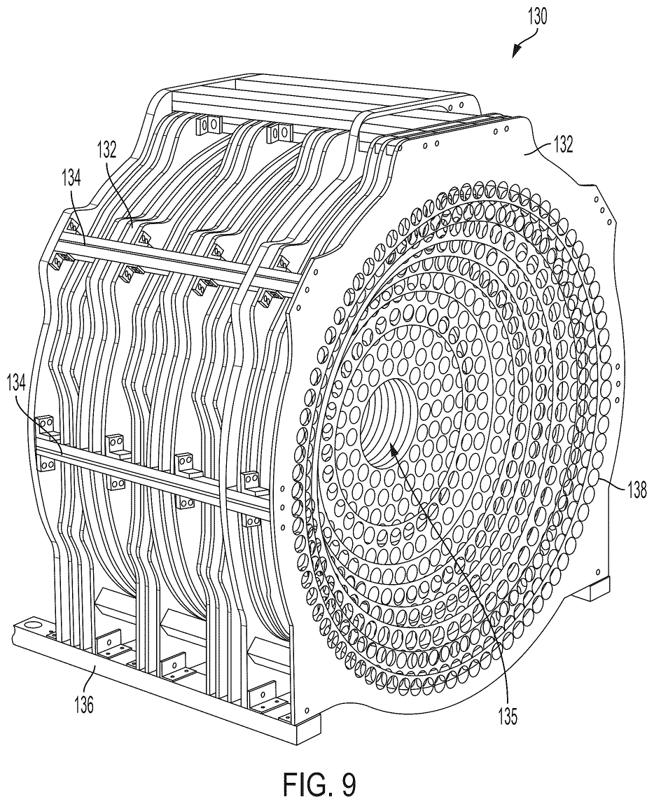

Referring primarily to FIGS. 7-9, the permanent magnet assembly 130 includes an array of magnets. The array of magnets forming the permanent magnet assembly 130 are configured to cover the front surface 125, or patient-facing surface, of the MRI scanner 100 (see FIG. 7) and are shown as horizontal bars in FIG. 8. The permanent magnet assembly 130 includes a plurality of cylindrical permanent magnets in a parallel configuration. Referring primarily to FIG. 9, the permanent magnet assembly 130 comprises parallel plates 132 that are held together by brackets 134. The system can be attached to the housing 120 of the MRI scanner 100 at a bracket 136. There can be a plurality of holes 138 in the parallel plates 132. The permanent magnet assembly 130 can include any suitable magnetic materials, including but not limited to rare-earth based magnetic materials, such as for example, Neodymium-based magnetic materials, for example.

The permanent magnet assembly 130 defines an access aperture or bore 135, which can provide access to the patient through the housing 120 from the opposite side of the housing 120. In other aspects of the present disclosure, the array of permanent magnets forming a permanent magnet assembly in the housing 120 may be bore-less and define an uninterrupted or contiguous arrangement of permanent magnets without a bore defined therethrough. In still other instances, the array of permanent magnets in the housing 120 may form more than one bore/access aperture therethrough.

In accordance with various aspects of the present disclosure, the permanent magnet assembly 130 provides a magnetic field B0 in a region of interest 190 that is along the Z axis, shown in FIG. 5. The Z axis is perpendicular to the permanent magnet assembly 130. Stated differently, the Z axis extends from a center of the permanent magnet assembly 130 and defines a direction of the magnetic field B0 away from the face of the permanent magnet assembly 130. The Z axis can define the primary magnetic field B0 direction. The primary magnetic field B0 can decrease along the Z axis, i.e. an inherent gradient, farther from the face of the permanent magnet assembly 130 and in the direction indicated with the arrow in FIG. 5.

In one aspect, the inhomogeneity of the magnetic field in the region of interest 190 for the permanent magnet assembly 130 can be approximately 81,000 ppm. In another aspect, the inhomogeneity of the magnetic field strength in the region of interest 190 for the permanent magnet assembly 130 can be between 200 ppm to 200,000 ppm and can be greater than 1,000 ppm in certain instances, and greater than 10,000 ppm in various instances.

In one aspect, the magnetic field strength of the permanent magnet assembly 130 can be less than 1 T. In another aspect, the magnetic field strength of the permanent magnet assembly 130 can be less than 0.5 T. In other instances, the magnetic field strength of the permanent magnet assembly 130 can be greater than 1 T and may be 1.5 T, for example. Referring primarily to FIG. 5, the Y axis extends up and down from the Z axis and the X axis extends to the left and right from the Z axis. The X axis, the Y axis, and the Z axis are all orthogonal to one another and the positive direction of each axis is indicated by the corresponding arrow in FIG. 5.

The RF transmission coils 140 may be configured to transmit RF waveforms and associated electromagnetic fields. The RF pulses from the RF transmission coils 140 may be configured to rotate the magnetization produced by the permanent magnet 130 by generating an effective magnetic field, referred to as B1, that is orthogonal to the direction of the permanent magnetic field (e.g. an orthogonal plane).

Referring primarily to FIG. 7, the gradient coil set 150 may include two sets of gradient coils 152, 154. The sets of gradient coils 152, 154 may be positioned on the face or front surface 125 of the permanent magnet assembly 130 intermediate the permanent magnet assembly 130 and the region of interest 190. Each set of gradient coils 152, 154 may include a coil portion on opposing sides of the bore 135. Referring to the axes in FIG. 5, the gradient coil set 154 may be the gradient coil set corresponding to the X axis, for example, and the gradient coil set 152 may be the gradient coil set corresponding to the Y axis, for example. The gradient coils 152, 154 can enable encoding along the X axis and Y axis, as further described herein.

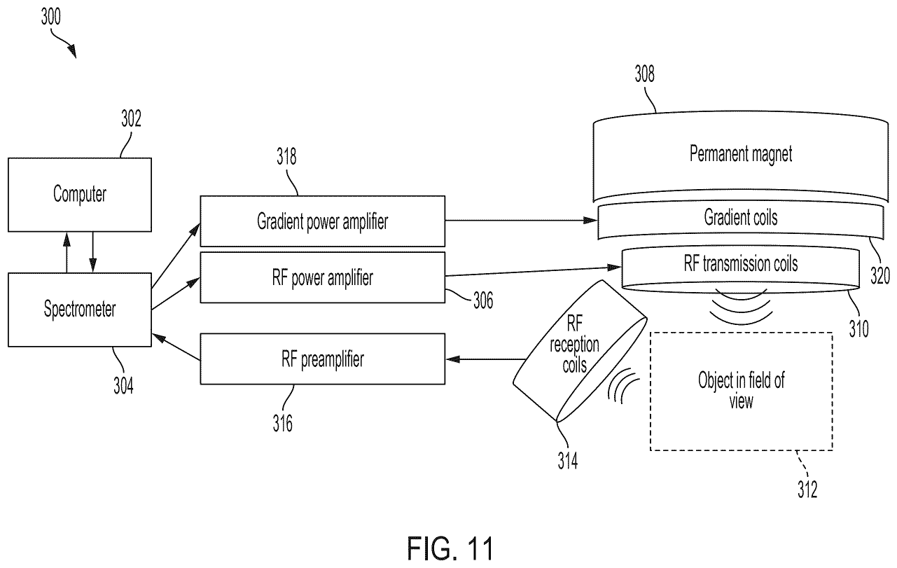

Referring now to FIG. 11, a control schematic for a single-sided MRI system 300 is shown. The single-sided MRI scanner 100 and/or components thereof (FIGS. 5-10) can be incorporated into the MRI system 300 in various aspects of the present disclosure. For example, the imaging system 300 includes a permanent magnet assembly 308, which can be similar to the permanent magnet assembly 130 (see FIGS. 6-9) in various instances. The imaging system 300 may also include RF transmission coils 310, which can be similar to the RF transmission coil 140 (see FIG. 7), for example. Moreover, the imaging system 300 may include RF reception coils 314, which can be similar to the RF reception coils 170 (see FIG. 7), for example. In various aspects, the RF transmission coils 310 and/or the RF reception coils can also be positioned in the housing of an MRI scanner and, in certain instances, the RF transmission coils 310 and the RF reception coils 314 can be combined into integrated Tx/Rx coils. The system 300 may also include gradient coils 320, which are configured to generate gradient fields to facilitate imaging of the object in the field of view 312.

The single-sided MRI system 300 may also include a computer 302, which is in signal communication with a spectrometer 304, and is configured to send and receive signals between the computer 302 and the spectrometer 304.

The main magnetic field BO generated by the permanent magnet 308 may extend away from the permanent magnet 308 and away from the RF transmission coils 310 into the field of view 312. The field of view 312 may contain an object that is being imaged by the MRI system 300.

During the imaging process, the main magnetic field B0 may extend into the field of view 312. The direction of the effective magnetic field (B1) may change in response to the RF pulses and associated electromagnetic fields from the RF transmission coils 310. For example, the RF transmission coils 310 are configured to selectively transmit RF signals or pulses to an object in the field of view, e.g. tissue. These RF pulses can alter the effective magnetic field experienced by the spins in the sample (e.g. patient tissue). When the RF pulses are on, the effective field experienced by spins on resonance may solely be the RF pulse, effectively canceling the static B0 field. The RF pulses can be chirp or frequency sweep pulses, for example, as further described herein.

Moreover, when the object in the field of view 312 is excited with RF pulses from the RF transmission coils 310, the precession of the object can result in an induced electric current, or MR current, which is detected by the RF reception coils 314. The RF reception coils 314 can send the excitation data to an RF preamplifier 316. The RF preamplifier 316 can boost or amplify the excitation data signals and send them to the spectrometer 304. The spectrometer 304 can send the excitation data to the computer 302 for storage, analysis, and image construction. The computer 302 can combine multiple stored excitation data signals to create an image, for example.

From the spectrometer 304, signals can also be relayed to the RF transmission coils 310 via an RF power amplifier 306, and to the gradient coils 320 via a gradient power amplifier 318. The RF power amplifier 306 may amplify the signal and sends it to RF transmission coils 310. The gradient power amplifier 318 may amplify the gradient coil signal and send it to the gradient coils 320.

Systems and methods for effectively collecting nuclear magnetic resonance spectra and magnetic resonance images in inhomogeneous fields, such as with the single-sided MRI scanner 100 and system 300, for example, are described herein.

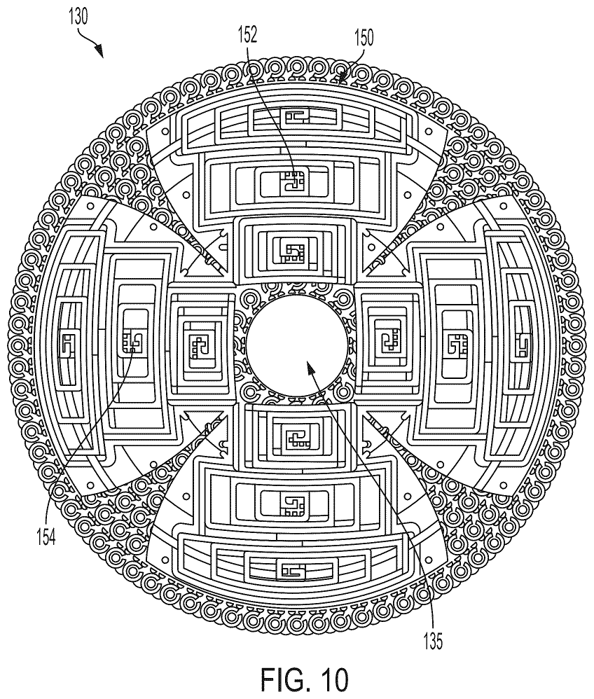

Imaging with a single-sided or open MRI can present many challenges. Typically, two sets of gradient coils (see FIG. 10) in single-sided systems are placed on the face of the permanent magnet assembly. As a result, the amplitude of the gradient may drop as one moves away from the face of the permanent magnet assembly. So, for a given array of phase encodes, the field of view may change as one moves along the axis of the permanent magnetic field B0. In other words, the pulsed gradient coils in a single-sided scanner can have a small component along the direction of the permanent gradient.

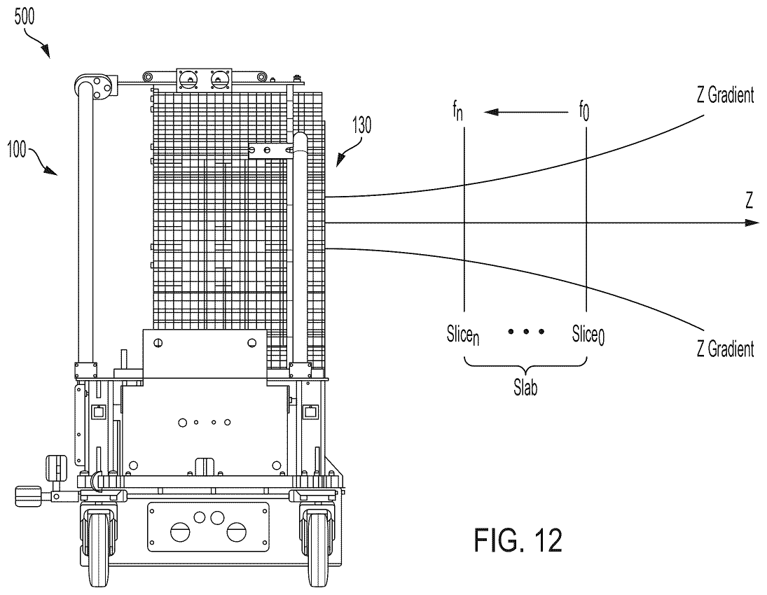

FIG. 12 is a schematic 500 of the magnetic field gradient along the Z axis for the MRI scanner 100. The permanent magnet 130 may have an inherent gradient along the Z axis. The strength of the Z gradient may decrease as one moves away from the permanent magnet 130. The Z gradient can be seen in the schematic bending away as one moves away from the permanent magnet causing the strength of the gradient to decrease. The MRI scanner 100 may image multiple slices to create a slab. Each slice may be excited for imaging at a different frequency. The lower frequencies may excite tissue for slices farther away from the permanent magnet and higher frequencies may excite the tissue in slices closer to the magnet. In the schematic, the slab or axial image is made of multiple slices going from Slice₀ to Sliceₙ. Each slice can have a corresponding frequency f₀ to fₙ, where f₀ is a frequency that is smaller than fₙ.

In accordance with various aspects of the present disclosure, it is possible to compensate for added phase by applying a phase encode during a frequency sweep, or chirped, excitation pulse. A frequency sweep pulse can affect spins at different frequencies at different times during a pulse. This means that it may also be possible to impart different amounts of phase to different frequencies by applying a phase encode during an excitation pulse. The spins excited at the beginning of the pulse can accumulate more phase than the spins excited at the end of the pulse, which can accumulate little phase.

In accordance with various aspects, if the spins further from the permanent magnet are excited first, and if a phase encode is applied during the frequency sweep excitation pulse, then those farther away spins can accumulate more phase than the spins closer to the permanent magnet, which can be excited last. This can invert the usual way spins accumulate phase from a surface gradient coil, allowing one to counter the normal variation in gradient strength along the Z axis. By precisely tuning the amount of phase accumulated during the frequency sweep excitation and during a subsequent phase encode, it may be possible to apply an even amount of phase to the X-Y plane along the Z axis of the permanent magnet.

### EXAMPLES

Various aspects of the subject matter described herein are set out in the following numbered examples.

Example 1—A method of identifying a region of interest using a low-field magnetic resonance imaging (MRI) system. The method comprising obtaining a T2-weighted image from the low-field MRI system, wherein the T2-weighted image comprises a slice, annotating a first region on the slice, wherein the first region corresponds to a suspicious region, and annotating a second region on the slice, wherein the second region corresponds to a non-suspicious region. The second region comprises the same size as the first region. The method further comprises computing a first texture feature value for the first region, computing a second texture feature value for the second region, and comparing the first texture feature value to the second texture feature value.

Example 2—The method of Example 1, wherein the first texture feature value and the second texture feature value correspond to a Haralick texture feature selected from a group consisting of Energy, Homogeneity, Contrast, and Correlation.

Example 3—The method of Examples 1 or 2, further comprising generating a graphical representation comparing the first texture feature value to the second texture feature value.

Example 4—The method of Example 1, 2, or 3, further comprising computing a plurality of first texture feature values for the first region, and computing a plurality of second texture feature values for the second region, wherein the plurality of first texture feature values and second texture feature values correspond to Haralick texture features selected from a group consisting of Energy, Homogeneity, Contrast, and Correlation.

Example 5—The method of Examples 1, 2, 3, or 4, further comprising generating a gray level co-occurrence matrix for the slice.

Example 6—The method of Example 5, wherein the gray level co-occurrence matrix is calculated with between 4 and 256 bins.

Example 7—The method of Examples 5 or 6, further comprising generating a texture map from the gray level co-occurrence matrix.

Example 8—The method of Examples, 5, 6, or 7, wherein computing the first texture feature value comprises calculating an average first value using a sliding window technique in the gray level co-occurrence matrix.

Example 9—The method of Example 8, wherein the sliding window technique comprises a sliding window size between 5 by 5 pixels and 49 by 49 pixels, and wherein the sliding window technique further comprises a sliding window stride between one and ten pixels.

Example 10—A system, comprising a single-sided, low-field MRI system comprising an array of magnets configured to generate a permanent, non-uniform B0 magnetic field in a region of interest offset from the array of magnets, and a control circuit. The control circuit configured to generate a T2-weighted image from the single-sided, low-field MRI, identify a first region on the T2-weighted image, wherein the first region corresponds to a suspicious region, and identify a second region on the T2-weighted image. The second region corresponds to a non-suspicious region, and wherein the second region comprises the same size as the first region. The control circuit configured to compute a first texture feature value for the first region, compute a second texture feature value for the second region, and comparing the first texture feature value to the second texture feature value. The system further comprising a display configured to convey the comparison of the first texture feature value to the second texture feature value.

Example 11—The system of Example 10, wherein the single-sided, low-field MRI system further comprises a housing comprising a face, wherein a first axis extends through the face into the region of interest, and wherein the permanent, non-uniform B0 magnetic field extends from the array of permanent magnets relative to the first axis into the region of interest.

Example 12—The system Examples 10 or 11, wherein the permanent, non-uniform B0 magnetic field comprises a magnetic field strength of less than 100 mT in the region of interest.

Example 13—The system of Examples 10 or 11, wherein the permanent, non-uniform B0 magnetic field comprises a magnetic field strength between 58 mT and 74 mT in the region of interest.

Example 14—The system of Examples 10, 11, 12, or 13, wherein the single-sided, low-field MRI system further comprises: a gradient coil set, at least one radio frequency coil, a power circuit, and a memory, wherein the control circuit is in signal communication with the gradient coil set, the at least one radio frequency coil, the power circuit, and the memory.

Example 15—The system of Examples 10, 11, 12, 13, or 14, wherein the first texture feature value and the second texture feature value correspond to a Haralick texture feature selected from a group consisting of Energy, Homogeneity, Contrast, and Correlation.

Example 16—The system of Examples 10, 11, 12, 13, 14, or 15, wherein the control circuit is further configured to compute a plurality of texture feature values for the first region, and compute a plurality of texture feature values for the second region. The plurality of texture feature values correspond to Haralick texture features selected from a group consisting of Energy, Homogeneity, Contrast, and Correlation.

Example 17—The system of Examples 10, 11, 12, 13, 14, 15, or 16, wherein the comparison comprises a graphical representation.

Example 18—The system of Examples 10, 11, 12, 13, 14, 15, 16, or 17, wherein the control circuit is further configured to generate a gray level co-occurrence matrix, and wherein the gray level co-occurrence matrix is calculated with between 4 and 256 bins.

Example 19—The system of Example 18, wherein the control circuit is further configured to generate a texture map from the gray level co-occurrence matrix, and calculate an average first value using a sliding window technique in the gray level co-occurrence matrix. The sliding window technique comprises a sliding window size between 5 by 5 pixels and 49 by 49 pixels, and wherein the sliding window technique further comprises a sliding window stride between one and ten pixels.

While several forms have been illustrated and described, it is not the intention of Applicant to restrict or limit the scope of the appended claims to such detail. Numerous modifications, variations, changes, substitutions, combinations, and equivalents to those forms may be implemented and will occur to those skilled in the art without departing from the scope of the present disclosure. Moreover, the structure of each element associated with the described forms can be alternatively described as a means for providing the function performed by the element. Also, where materials are disclosed for certain components, other materials may be used. It is therefore to be understood that the foregoing description and the appended claims are intended to cover all such modifications, combinations, and variations as falling within the scope of the disclosed forms. The appended claims are intended to cover all such modifications, variations, changes, substitutions, modifications, and equivalents.

The foregoing detailed description has set forth various forms of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, and/or examples can be implemented, individually and/or collectively, by a wide range of hardware, software, firmware, or virtually any combination thereof. Those skilled in the art will recognize that some aspects of the forms disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as one or more program products in a variety of forms, and that an illustrative form of the subject matter described herein applies regardless of the particular type of signal bearing medium used to actually carry out the distribution.

Instructions used to program logic to perform various disclosed aspects can be stored within a memory in the system, such as dynamic random access memory (DRAM), cache, flash memory, or other storage. Furthermore, the instructions can be distributed via a network or by way of other computer readable media. Thus a machine-readable medium may include any mechanism for storing or transmitting information in a form readable by a machine (e.g., a computer), but is not limited to, floppy diskettes, optical disks, compact disc, read-only memory (CD-ROMs), and magneto-optical disks, read-only memory (ROMs), random access memory (RAM), erasable programmable read-only memory (EPROM), electrically erasable programmable read-only memory (EEPROM), magnetic or optical cards, flash memory, or a tangible, machine-readable storage used in the transmission of information over the Internet via electrical, optical, acoustical or other forms of propagated signals (e.g., carrier waves, infrared signals, digital signals, etc.). Accordingly, the non-transitory computer-readable medium includes any type of tangible machine-readable medium suitable for storing or transmitting electronic instructions or information in a form readable by a machine (e.g., a computer).

As used in any aspect herein, the term “control circuit” may refer to, for example, hardwired circuitry, programmable circuitry (e.g., a computer processor including one or more individual instruction processing cores, processing unit, processor, microcontroller, microcontroller unit, controller, digital signal processor (DSP), programmable logic device (PLD), programmable logic array (PLA), or field programmable gate array (FPGA)), state machine circuitry, firmware that stores instructions executed by programmable circuitry, and any combination thereof. The control circuit may, collectively or individually, be embodied as circuitry that forms part of a larger system, for example, an integrated circuit (IC), an application-specific integrated circuit (ASIC), a system on-chip (SoC), desktop computers, laptop computers, tablet computers, servers, smart phones, etc. Accordingly, as used herein “control circuit” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

As used in any aspect herein, the term “logic” may refer to an app, software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices.

As used in any aspect herein, the terms “component,” “system,” “module” and the like can refer to a computer-related entity, either hardware, a combination of hardware and software, software, or software in execution.

As used in any aspect herein, an “algorithm” refers to a self-consistent sequence of steps leading to a desired result, where a “step” refers to a manipulation of physical quantities and/or logic states which may, though need not necessarily, take the form of electrical or magnetic signals capable of being stored, transferred, combined, compared, and otherwise manipulated. It is common usage to refer to these signals as bits, values, elements, symbols, characters, terms, numbers, or the like. These and similar terms may be associated with the appropriate physical quantities and are merely convenient labels applied to these quantities and/or states.

A network may include a packet switched network. The communication devices may be capable of communicating with each other using a selected packet switched network communications protocol. One example communications protocol may include an Ethernet communications protocol which may be capable permitting communication using a Transmission Control Protocol/Internet Protocol (TCP/IP). The Ethernet protocol may comply or be compatible with the Ethernet standard published by the Institute of Electrical and Electronics Engineers (IEEE) titled “IEEE 802.3 Standard”, published in December 2008 and/or later versions of this standard. Alternatively or additionally, the communication devices may be capable of communicating with each other using an X.25 communications protocol. The X.25 communications protocol may comply or be compatible with a standard promulgated by the International Telecommunication Union-Telecommunication Standardization Sector (ITU-T). Alternatively or additionally, the communication devices may be capable of communicating with each other using a frame relay communications protocol. The frame relay communications protocol may comply or be compatible with a standard promulgated by Consultative Committee for International Telegraph and Telephone (CCITT) and/or the American National Standards Institute (ANSI). Alternatively or additionally, the transceivers may be capable of communicating with each other using an Asynchronous Transfer Mode (ATM) communications protocol. The ATM communications protocol may comply or be compatible with an ATM standard published by the ATM Forum titled “ATM-MPLS Network Interworking 2.0” published August 2001, and/or later versions of this standard. Of course, different and/or after-developed connection-oriented network communication protocols are equally contemplated herein.

Unless specifically stated otherwise as apparent from the foregoing disclosure, it is appreciated that, throughout the foregoing disclosure, discussions using terms such as “processing,” “computing,” “calculating,” “determining,” “displaying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

One or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

The terms “proximal” and “distal” are used herein with reference to a clinician manipulating the handle portion, or housing, of a surgical instrument. The term “proximal” refers to the portion closest to the clinician and/or to the robotic arm and the term “distal” refers to the portion located away from the clinician and/or from the robotic arm. It will be further appreciated that, for convenience and clarity, spatial terms such as “vertical”, “horizontal”, “up”, and “down” may be used herein with respect to the drawings. However, robotic surgical tools are used in many orientations and positions, and these terms are not intended to be limiting and/or absolute.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flow diagrams are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

It is worthy to note that any reference to “one aspect,” “an aspect,” “an exemplification,” “one exemplification,” and the like means that a particular feature, structure, or characteristic described in connection with the aspect is included in at least one aspect. Thus, appearances of the phrases “in one aspect,” “in an aspect,” “in an exemplification,” and “in one exemplification” in various places throughout the specification are not necessarily all referring to the same aspect. Furthermore, the particular features, structures or characteristics may be combined in any suitable manner in one or more aspects.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

In summary, numerous benefits have been described which result from employing the concepts described herein. The foregoing description of the one or more forms has been presented for purposes of illustration and description. It is not intended to be exhaustive or limiting to the precise form disclosed. Modifications or variations are possible in light of the above teachings. The one or more forms were chosen and described in order to illustrate principles and practical application to thereby enable one of ordinary skill in the art to utilize the various forms and with various modifications as are suited to the particular use contemplated. It is intended that the claims submitted herewith define the overall scope.

1. A method of identifying a region of interest using a low-field magnetic resonance imaging (MRI) system, the method comprising:
obtaining a T2-weighted image from the low-field MRI system, wherein the T2-weighted image comprises a slice;
annotating a first region on the slice, wherein the first region corresponds to a suspicious region;
annotating a second region on the slice, wherein the second region corresponds to a non-suspicious region, and wherein the second region comprises the same size as the first region;
computing a first texture feature value for the first region;
computing a second texture feature value for the second region; and
comparing the first texture feature value to the second texture feature value.

2. The method of claim 1, wherein the first texture feature value and the second texture feature value correspond to a Haralick texture feature selected from a group consisting of Energy, Homogeneity, Contrast, and Correlation.

3. The method of claim 1, further comprising generating a graphical representation comparing the first texture feature value to the second texture feature value.

4. The method of claim 1, further comprising:
computing a plurality of first texture feature values for the first region; and
computing a plurality of second texture feature values for the second region, wherein the plurality of first texture feature values and second texture feature values correspond to Haralick texture features selected from a group consisting of Energy, Homogeneity, Contrast, and Correlation.

5. The method of claim 1, further comprising generating a gray level co-occurrence matrix for the slice.

6. The method of claim 5, wherein the gray level co-occurrence matrix is calculated with between 4 and 256 bins.

7. The method of claim 5, further comprising generating a texture map from the gray level co-occurrence matrix.

8. The method of claim 7, wherein computing the first texture feature value comprises calculating an average first value using a sliding window technique in the gray level co-occurrence matrix.

9. The method of claim 8, wherein the sliding window technique comprises a sliding window size between 5 by 5 pixels and 49 by 49 pixels, and wherein the sliding window technique further comprises a sliding window stride between one and ten pixels.

10. A system, comprising:
a single-sided, low-field MRI system comprising an array of magnets configured to generate a permanent, non-uniform BO magnetic field in a region of interest offset from the array of magnets;
a control circuit configured to:
generate a T2-weighted image from the single-sided, low-field MRI;
identify a first region on the T2-weighted image, wherein the first region corresponds to a suspicious region;
identify a second region on the T2-weighted image, wherein the second region corresponds to a non-suspicious region, and wherein the second region comprises the same size as the first region;
compute a first texture feature value for the first region;
compute a second texture feature value for the second region; and
compare the first texture feature value to the second texture feature value; and
a display configured to convey the comparison of the first texture feature value to the second texture feature value.

11. The system of claim 10, wherein the single-sided, low-field MRI system further comprises a housing comprising a face, wherein a first axis extends through the face into the region of interest, and wherein the permanent, non-uniform B0 magnetic field extends from the array of permanent magnets relative to the first axis into the region of interest.

12. The system of claim 10, wherein the permanent, non-uniform B0 magnetic field comprises a magnetic field strength of less than 100 mT in the region of interest.

13. The system of claim 10, wherein the permanent, non-uniform B0 magnetic field comprises a magnetic field strength between 58 mT and 74 mT in the region of interest.

14. The system of claim 10, wherein the single-sided, low-field MRI system further comprises:
a gradient coil set;
at least one radio frequency coil;
a power circuit; and
a memory;
wherein the control circuit is in signal communication with the gradient coil set, the at least one radio frequency coil, the power circuit, and the memory.

15. The system of claim 10, wherein the first texture feature value and the second texture feature value correspond to a Haralick texture feature selected from a group consisting of Energy, Homogeneity, Contrast, and Correlation.

16. The system of claim 10, wherein the control circuit is further configured to:
compute a plurality of texture feature values for the first region; and
compute a plurality of texture feature values for the second region, wherein the plurality of texture feature values correspond to Haralick texture features selected from a group consisting of Energy, Homogeneity, Contrast, and Correlation.

17. The system of claim 10, wherein the comparison comprises a graphical representation.

18. The system of claim 10, wherein the control circuit is further configured to generate a gray level co-occurrence matrix, and wherein the gray level co-occurrence matrix is calculated with between 4 and 256 bins.

19. The system of claim 18, wherein the control circuit is further configured to:
generate a texture map from the gray level co-occurrence matrix; and
calculate an average first value using a sliding window technique in the gray level co-occurrence matrix, wherein the sliding window technique comprises a sliding window size between 5 by 5 pixels and 49 by 49 pixels, and wherein the sliding window technique further comprises a sliding window stride between one and ten pixels.
